(19) United States
(12) Patent Application Publication
(10) Pub. No.: US 2023/0366828 A1
(43) Pub. Date: Nov. 16, 2023

(54) BIOMARKER TEST WITH UNIFIED PIGMENTATION

(71) Applicants: Kiran R. Tremblay, Billerica, MA (US); Lonna Y. Neidig, Cambridge, MA (US); Alexandra M. Gannon, Fremont, NH (US); Burnon J. Chen, Marlborough, MA (US)

(72) Inventors: Kiran R. Tremblay, Billerica, MA (US); Lonna Y. Neidig, Cambridge, MA (US); Alexandra M. Gannon, Fremont, NH (US); Burnon J. Chen, Marlborough, MA (US)

(21) Appl. No.: 18/135,882

(22) Filed: Apr. 18, 2023

Related U.S. Application Data
(60) Provisional application No. 63/332,046, filed on Apr. 18, 2022.

(51) Int. Cl. G01N 21/78; G01N 33/573; G01N 33/70

(52) U.S. Cl. CPC G01N 21/78; G01N 33/573; G01N 33/70; G01N 2333/912

(57) ABSTRACT

An inexpensive test device compares and contrasts a plurality of pigmented tests for a biomarker, organism or other substance for a relative presence of each. Each biomarker is present in a urine sample, and the pigmented test renders a color shade indicative of a respective proportion or quantity. Collection of the pigmented test results in a series of transparent vials allowing a common line of sight through each vial to result in an appearance of a blended or combined color, similar to viewing color filters in line. A predetermined control group of color combinations provides a comparison for respective concentrations of each biomarker present in the sample. In one approach, a first pigmented test results in an orange-yellow shade and a second pigmented test results in a blue shade that, when viewed inline, appear as a green, brown or purple shade indicative of relative percentages denoted by each respective test.

### RELATED APPLICATIONS

This patent application claims the benefit under 35 U.S.C. § 119(e) of U.S. Provisional Patent App. No. 63/332,046, filed Apr. 18, 2022, entitled “MULTIPLE BIOMARKER TEST WITH UNIFIED PIGMENTATION,” incorporated herein by reference in entirety.

### BACKGROUND

Endometriosis is an elusive condition which is generally invasive and expensive to detect clinically. Conventional noninvasive diagnostic procedures employing medical imaging are expensive and often inconclusive. Pelvic examination, ultrasound and Magnetic Resonance Imaging (MRI) can be employed, but conventional test results may be inconclusive without a laparoscopic surgical procedure for internal examination. Endometriosis causes a chronic inflammatory reaction that may result in painful internal lesions and formation of scar tissue within the pelvis and other parts of the body.

### SUMMARY

An inexpensive test device compares and contrasts a plurality of pigmented tests for a biomarker, organism or other substance for a relative presence of each. Each biomarker is present in a urine sample, and the pigmented test renders a color shade indicative of a respective proportion or quantity. Collection of the pigmented test results in a series of transparent vials allowing a common line of sight through each vial to result in an appearance of a blended or combined color, similar to viewing color filters in line. An LED placed behind the vials provides a uniform backlight that allows for consistent and reproducible visual results. A predetermined control group of color combinations provides a comparison for respective concentrations of each biomarker present in the sample. In one approach, a first pigmented test results in an orange-yellow shade and a second pigmented test results in a blue shade that, when viewed inline, appear as a green, brown or purple shade indicative of relative percentages of a respective biomarker denoted by each test.

Configurations herein are based, in part, on the observation that colorimetric, medical tests facilitate detection of certain health related substances based on a presence in a readily available bodily sample, such as urine, blood and saliva. Unfortunately, conventional approaches to at-home testing are often limited to a single vial sample or testing exchange with a reactive substance. Tests which require consideration of a relative substance quantity dependent on one or more other substances are often excessively complex or expensive for wide scale distribution. Accordingly, configurations herein substantially overcome the shortcomings of conventional approaches by providing a dual vial colorimetric test which provides a combined shade by viewing both vials simultaneously to arrive at a test result based on relative percentages of multiple biomarkers or substances where a mere indication of one biomarker is indeterminate without a level of another biomarker or substance.

In a particular configuration, a method for detecting a ratio of biomarkers includes generating a plurality of reactions in a respective plurality of containments, where each reaction of the plurality of reactions is based on a pigmented test agent indicative of a presence of a biomarker. The test procedure forms a shade in each of the containments resulting from a concentration of the respective biomarker, and the containments are transparent for visualization of the shade. Disposing the containments in an optical adjacency, such as inline through each transparent containment, generates a combined shade based on each respective shade, the combined shade indicative of a ratio of each of the biomarkers.

A particular configuration is directed to testing of endometriosis using a colorimetric test for soluble Fms-Like Tyrosine Kinase-1, or SFLT-1, to produce a blue shade aligned with a yellow-orange shade resulting from an established creatinine test. The test for the SFLT-1 biomarker includes coating a surface with a binding protein, and adhering an antibody of the biomarker to the binding protein. A test specimen, typically urine, containing the biomarker is combined with the antibody of the biomarker for binding the biomarker to the antibody. A subcomplex is conjugated including an ALP (alkaline phosphatase) bound VEGF (vascular endothelial growth factor) compound, and combined with the bound test biomarker for forming a color complex. A pigmented test agent including an ALP substrate and having an affinity for the color complex is added to generate a pigment indicative of the biomarker, resulting in a blue shade based on the SFLT-1 biomarker. The relative presence of the biomarker is visualized based on a visual shade resulting from the generated pigment indicative of a ratio of both creatinine and SFLT-1.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a system context diagram of an endometriosis test device depicting a dual blended color rendering for indicating a ratio of two biomarker substances present in a urine sample based on first and second vials of respective pigmented test agents;

FIG. 2 shows a second vial in the device of FIG. 1 for forming a first complex adapted for SFLT-1 detection;

FIG. 3 shows binding of urinary SFLT-1 in the second vial of FIG. 2;

FIG. 4 shows binding of a second complex in the second vial;

FIG. 5 shows introduction of an ALP substrate defining a pigmented test agent for SFLT-1 in the second vial;

FIG. 6 shows binding of the pigmented test agent as in FIG. 5 to form a color factory defined by the ALP substrate;

FIG. 7 shows pigment generation over time of the pigmented test agent of FIG. 6;

FIGS. 8A and 8B show an alternate configuration using biotinylation for augmenting pigment generation in the pigmented test agent of FIGS. 6 and 7; and

FIGS. 9A and 9B show an example of the device including the vials and color scale indicative of the endometriosis diagnosis result.

### DETAILED DESCRIPTION

Urinalysis provides an inexpensive test for physiological indicators of health conditions for substances that are excreted via the kidneys in the urine stream. Tests targeted at urine bound substances, combined with a pigmented test agent, can render a shade indicative of the urine bound substance under test. Alkaline Phosphate (ALP) is one such test agent that can be used to generate or form a visible pigment coloration in a shade indicative of a presence of the substance under test, typically in a color depth proportional to the concentration of the substance under test. A problem exists in determining a pigmented test agent capable of binding to the substance under test. The description below presents a noninvasive urine test that indicates a potential presence and/or diagnosis of endometriosis, negating the need for invasive diagnostic procedures. Since the symptoms of endometriosis may also indicate other conditions, it can be difficult for doctors to determine the condition; this may lead to prolonged periods before a diagnosis can be made, and can potentially cause years of unnecessary suffering. This device can help diagnose endometriosis noninvasively with a simple urine test, eliminating the need for uncomfortable, extensive, and expensive physical examinations.

In the examples herein, a test device and method employs such pigmented tests for testing of endometriosis. In endometriosis detection, soluble Fms-Like Tyrosine Kinase-1, or SFLT-1, can be tested to ascertain and diagnose endometrioses.

According to John Hopkins Medicine, endometriosis affects between 2 to 10 percent of American women between the ages of 25 and 45, which is about 1 to 4 million women in the U.S. alone. Since diagnostic procedures are expensive, invasive, and often inconclusive, many women do not opt for testing or may not realize they have the disease. A noninvasive, quick, and inexpensive diagnostic test would greatly benefit potentially millions of patients.

A complication to the urinalysis approach results when a urinary presence of a substance under test is dependent on another urinary present substance. In the case of endometriosis, detection of SFLT-1 and creatinine are both relevant biomarkers in diagnosing endometriosis. Since SFLT-1 is only a significant biomarker when adjusted for the concentration of creatinine, both SFLT-1 and creatinine must be detected quantifiably. Thus, a method was devised to detect both analytes using colored pigments. Creatinine can be detected quantitatively using the Jaffe reaction. A separate series of reactions and complexes were devised in order to quantitatively measure the amount of SFLT-1 in the urine and render this information using a colored pigment.

Accordingly, concentrations of SFLT-1 depend on the concentrations of creatinine in the urine, so both concentrations need to be measured in order to determine if the elevated SFLT-1 levels were caused by endometriosis. The creatinine concentration of the sample can be measured by matching the absorbance of the sample against a concentration-absorbance standard curve.

In an example configuration, a urine-based protein-screening test device includes a first vessel including a pigmented test agent for a first biomarker, and a second vessel including a pigmented test agent for a second biomarker, such that the first and second vessels are visually aligned for a common line of sight. The biomarker tests may include any test for a substance, organism or physiology based product that can be tested via pigmentation and is indicative of a physiologic condition or presence. A predetermined range of rendered colors indicates a blended shade and respective percentages of the biomarkers that they represent. The rendered colors are based on a combined shade resulting from a respective percentage of the first and second biomarkers, for example a green or brown-purple shade resulting from separate orange-yellow and blue test results. An LED placed behind the vials helps standardize visuals, as it provides a standard backlight for the colored vials.

The device may define a consumer product with a test vial assembly, such that the pigmented test agents are responsive to a respective biomarker for generating a pigmented shade having a color depth relative to the biomarker concentrations. The combined shade results from a blend of colors in a line of sight common to the first vessel and the second vessel, preferably against a white background and adjacent a concurrently viewed predetermined shade of result outcomes. Alternatively, an LED can be placed behind the device to provide a consistent backlight for the vials, and a wavelength filter tuned to a specific group of wavelengths can be placed between the viewer and the vials to aid in color-shade determination. It is therefore expected that the first vessel and the second vessel are transparent and adjacent for rendering a blended color based on a concurrent view of both the first and second vessel in a common line of sight. A wavelength filter or an adjacent background depicting a predetermined range of rendered colors that can be used for simultaneous presentation of the blended color can help assess color shade and diagnoses.

In the specific example herein for endometriosis, the pigmented test agent for the first biomarker includes a urinalysis test producing a pigmentation for a percentage of creatinine indicated by an orange-yellow coloration. The pigmented test agent for the second biomarker includes a first subcomplex defined by a surface coated with protein A/G and further combined with SFLT-1 antibodies, and a second subcomplex including an ALP-conjugated VEGF (vascular endothelial growth factor). Addition of an alkaline phosphatase (ALP) substrate adapted for a time based pigmentation based on a presence of SFLT-1 interacts with the first and second subcomplexes retained by the magnetic beads following removal of excess urine. This test indicates a percentage of SFLT-1 based on a blue coloration. The resulting combined shade has a green or brown-purple property resulting from an orange-yellow coloration and a blue coloration in the first and second vessels, respectively.

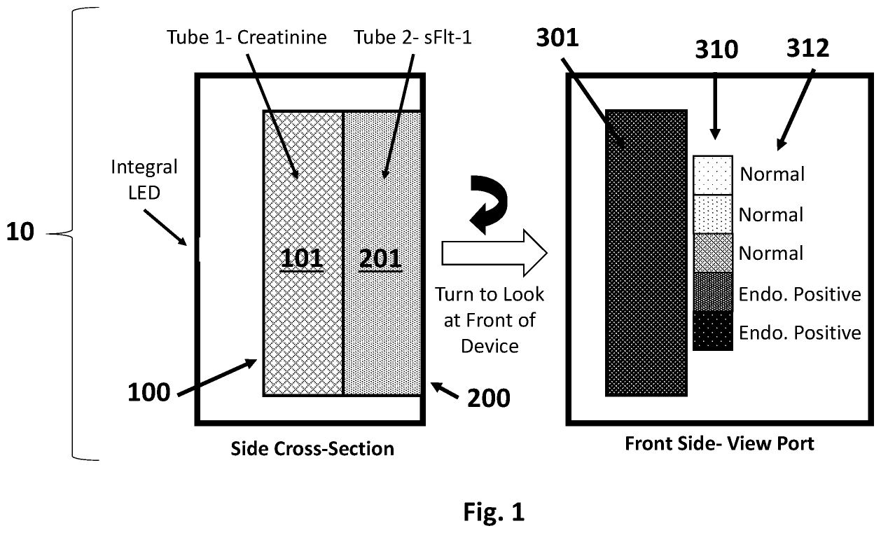

FIG. 1 is a system context diagram of an endometriosis test device 10 depicting a dual blended color rendering for indicating a ratio of two biomarker substances present in a urine sample based on first 100 and second 200 containment vials of respective pigmented test agents. In configurations herein, a method for detecting a ratio of biomarkers includes generating a plurality of reactions in a respective plurality of containments 100, 200 where each reaction of the plurality of reactions is based on a pigmented test agent indicative of a presence of a biomarker, in this case creatinine and SFLT-1. Substance specific tests, discussed below, form a respective shade 101, 201 in each of the containments 100, 200, where the shade results from a concentration of the respective biomarkers. The containments 100, 200 are transparent for visualization of the respective shades. The dual containment device 100 is such that by rotating the device a quarter turn disposes the containments in a line-of-sight optical adjacency for generating a combined shade 301 based on each respective shades 101, 201, where the combined shade 301 is indicative of a ratio of each of the biomarkers.

Viewing of the quarter-turned device 100 therefore renders a combined shade based on a line of sight through both of the adjacent transparent containments 100, 200. Alternatively, an image recognition camera and color detection processor could be employed to automate recognition of the combined shade. An adjacent color shade progression labels a diagnosis 312 of endometriosis, as the adjacent transparent containments 100, 200 are open for optical inspection for viewing the combined shade 301 adjacent to a color scale 310 indicative of a ratio of the first and second biomarkers.

Creatinine testing has been employed using a so-called Jaffe reaction for creatinine measurement using urinalysis methods. Creatinine reacted with picric acid in an alkaline environment, and developed using a base such as sodium hydroxide, forms a crystalline orange-yellow complex that precipitates into solution, forming a colored solution. The resulting color has a wavelength around 520 nanometers.

Certain protein detection methods use enzymes such as alkaline phosphatase or horseradish peroxidase to visually depict analyte concentrations. Alkaline phosphatase (ALP) is an enzyme that can be used to convert a colorless substrate into a colored substrate. Two common substrates of ALP create yellow and blue pigments respectively. Since the Jaffe reaction produces a yellow-orange colored pigment, a blue pigment produced by the ALP would produce an overall green or purple solution, which is ideal for identification. Since ALP acts as a “color-producing factory,” the longer the enzyme is active the more substrate it will produce. This allows for a quantifiable signal amplification, which means that the SFLT-1 will be able to be accurately detected and quantified even at low concentrations.

Blue-pigment producing ALP can be used to reliably and quantifiably determine the concentration of SFLT-1 in urine, provided that a complex can be developed that links a single ALP color factory to a single SFLT-1 molecule. If the device 100 utilizes complexes and reactions that ensure the only active ALP color factories are those tied to SFLT-1, then a 1:1 correlation can be formed between SFLT-1 molecules and active ALP conjugates. Thus, blue-pigment production can be tied to SFLT-1 concentration and can be corrected colorimetrically for creatinine concentration.

In the example of FIG. 1, a yellow orange shade 101 results from combining an acidic indicator with the test specimen in an alkaline environment, such that the acidic indicator is responsive to a presence of a second biomarker in the test specimen. The creatinine test in the first containment 100 thus employs picric acid for assessing creatinine via the orange/yellow shade 101.

The second containment 201 renders a blue shade 201, indicating a presence of SFLT-1, via a more complex reaction discussed below. The resulting combined shade 301 defines the purple-orange color scale 310 denoting a harmful presence of SFLT-1 311 based on the combined SFLT-1 and creatinine ratio.

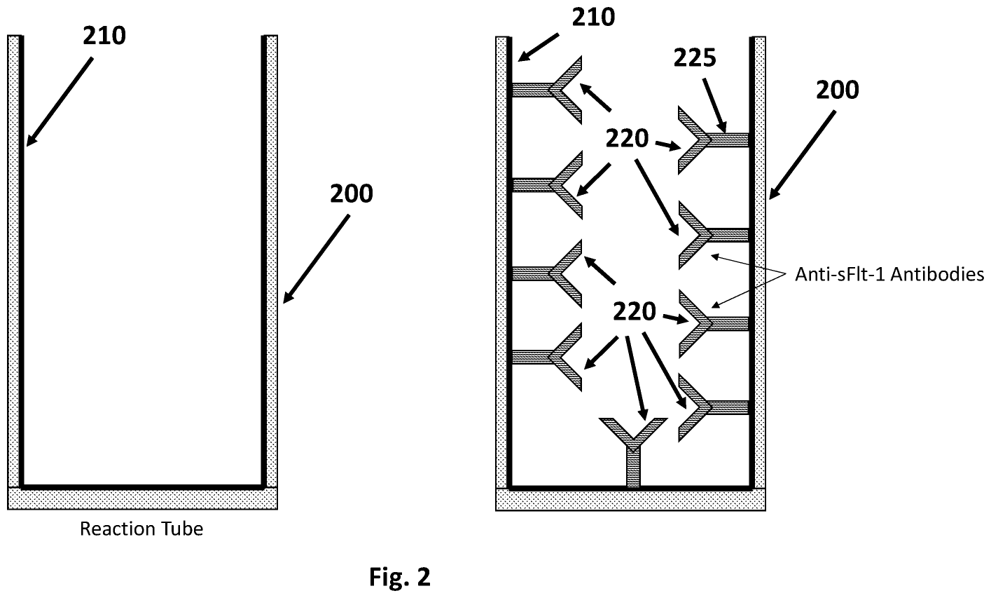

The second containment 201 occurs in a vial in several stages depicted below in FIGS. 2-7 for producing the blue shade 201. The formation and assembly of the blue-pigmented complex and accompanying reaction employs a variety of antibodies, proteins, and subcomplexes. An SFLT-1-VEGF-ALP conjugate complex is formed in-situ in the containment 200. The complete complex can be broken down into two subcomplexes and a “linker,” which will be SFLT-1 itself. A recombinant fusion protein such as protein A/G is introduced on a surface of the containment 200. Alternatively, magnetic beads coated with the protein A/G may be employed. The protein A/G will be combined with specific anti-SFLT-1 antibodies. These three components (the surface/bead, the protein A/G layer, and the anti-SFLT-1 antibodies) make up Subcomplex 1. These antibody-coated beads will bind to the urinary SFLT-1 upon contact with urine, essentially binding the SFLT-1 to the device. This establishes SFLT-1 as a linker. Subcomplex 2 consists of AP-conjugated VEGF. VEGF is a protein that binds to the active site of SFLT-1. Conjugating alkaline phosphatase to VEGF creates a subcomplex that allows the ALP to bind to the SFLT-1. When the subcomplexes come into contact with the linker (urinary SFLT-1) they create the total complex, which consists of ALP bound to VEGF, which is in turn bound to SFLT-1, which is bound to the anti-SFLT-1 antibody and is thus attached to the Protein A/G coating. Binding the SFLT-1 and ALP conjugated VEGF to the surface prevents the “color factory” from being accidentally washed out of the device. Removing the excess urine and unbound subcomplex 2 once the subcomplexes and SFLT-1 have finished combining is significant. Pouring out excess urine and VEGF-ALP conjugate ensures that only SFLT-1-bound-ALP actively produce pigment.

Following the interval for allowing the biomarkers in the test specimen to bind with the antibody, the remaining test specimen is removed from the containment 200. Once excess urine and subcomplex 2 have been rinsed from the device, the colorless ALP substrate can be introduced. At this point, the total complex has formed so that there is a single ALP color factory per SFLT-1 molecule bound to the device. Over time, the ALP converts the colorless substrate into a blue pigment. The concentration of blue pigment determines the shade of blue of the solution in the analysis tube. Since the rate of pigment production per ALP is relatively constant, the shade can be calibrated to a standard.

For a deliverable product including the device 10, since subcomplex 1 and subcomplex 2 cannot interact if there is no SFLT-1 present, and both complexes should be stable, they can be packaged into the device together. Once the device comes into contact with urine, the full “color factory” complex forms, and excess ALP can be rinsed out easily before the substrate is added. This is to ensure unbound ALP does not participate in substrate conversion into pigment.

FIG. 2 shows the second containment 201 vial in the device of FIG. 1 for forming a first complex adapted for SFLT-1 detection. Referring to FIG. 2, the containment 200 is coated with a binding protein 210 for receiving a liquid of the test specimen, typically a patient urine sample. In the example configuration, the transparent walls of the containment 200 are coated with Protein A/G, a nonspecific antibody-binding protein. The binding protein 210 is selected for adherence of an antibody 220 of the biomarker to the binding protein 210. The protein A/G allows for antibody binding to the walls of the reaction tube. For distribution of the device 100, the antibodies 220 would be attached to the device prior to use, forming a first complex 225 as referred to herein (subcomplex 1). Any suitable surface may suffice for the protein A/G coating, such as magnetic balls, which may facilitate alterative test containments.

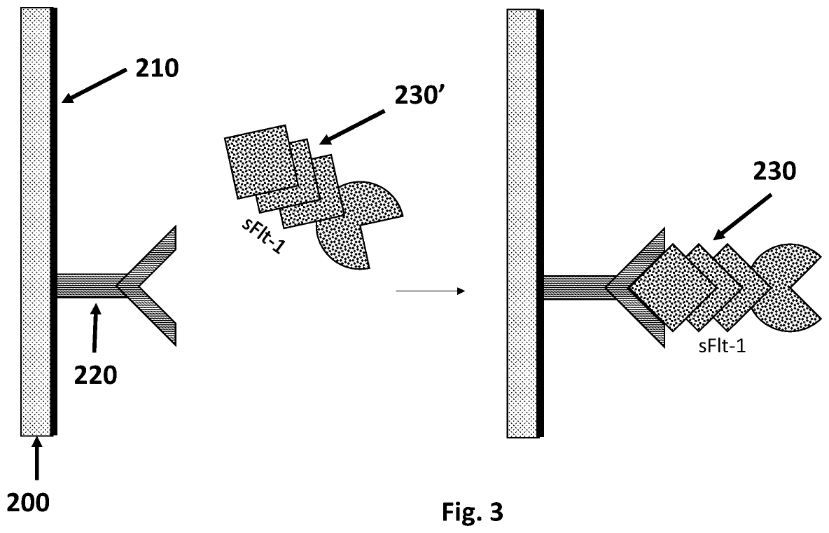

FIG. 3 shows binding of urinary SFLT-1 in the second vial of FIG. 2. Referring to FIGS. 2 and 3, a test specimen containing the biomarker combines with the antibody of the biomarker for binding the biomarker (SFLT-1 230) to the antibody 220. Upon introduction of the test specimen, SFLT-1 molecules 230′ tend to bind to their antibodies to form bound SFLT-1 230. When the urine test specimen is introduced to the reaction containment 200, the anti-SFLT-1 antibodies pull the SFLT-1 230′ out of solution and bind the biomarker proteins 230 to the wall of the device. Although SFLT-1 is shown as an example, other diagnostic processes could be performed by combining a test specimen containing the biomarker with the antibody of the biomarker for binding the biomarker to the antibody 220.

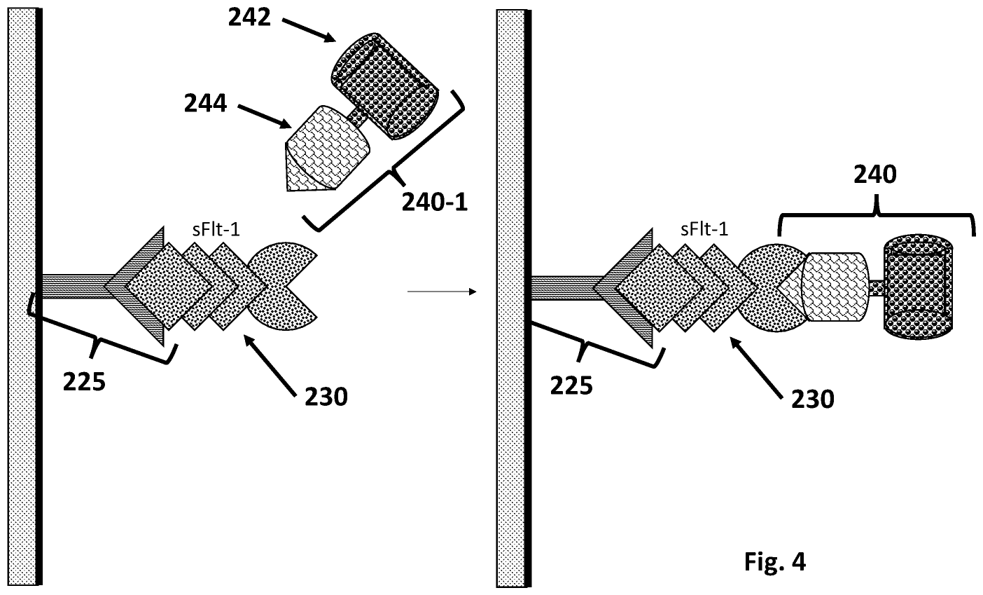

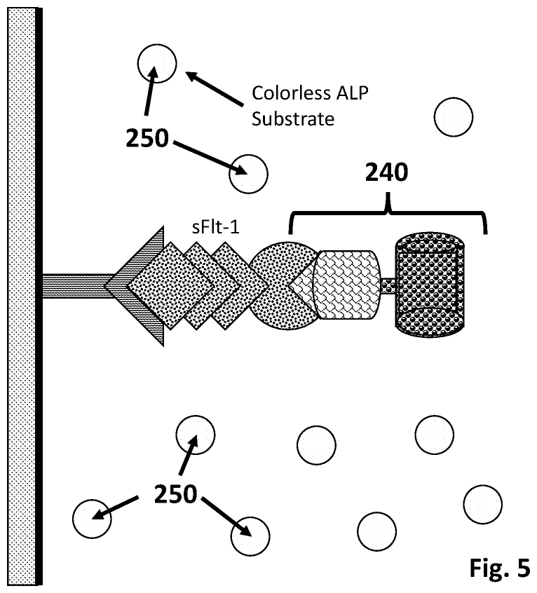

FIG. 4 shows binding of a second complex (subcomplex 2) in the second vial. After the first complex 225 is formed on a surface in the receptacle 200 (vial), the remaining urine is discarded as the SFLT-1 molecules remain bound to the first complex. The next step is to bind a conjugate including a protein and a coloring enzyme to the bound test biomarker (SFLT-1) for forming a second complex 240 (color complex) responsive to a pigmented test agent. The second complex is conjugated as a subcomplex including an ALP (alkaline phosphatase) 242 bound VEGF (vascular endothelial growth factor) 244 molecule and combining the subcomplex with the bound test biomarker 230 for forming a color complex. After a short amount of time for allowing the SFLT-1 to bind to the antibody in FIG. 3, the urine is removed from the containment 200 and a solution is added to the tube containing a complex of two proteins: Vascular endothelial growth factor and alkaline phosphatase (VEGF-ALP). This complex 240 then attaches to the SFLT-1 bound to the walls of the device, as VEGF is a high-affinity ligand of SFLT-1. Alternatively, the binding of the SFLT-1 to the antibody, and the subsequent binding of the ALP-VEGF could be performed with just the urine sample. Either way, after a short amount of time, the VEGF-ALP solution is rinsed out of the device, removing unbound VEGF-ALP complexes 240 in preparation for the reaction of FIG. 5.

FIG. 5 shows introduction of an ALP substrate defining a pigmented test agent for SFLT-1 in the second containment 200. Once the VEGF-ALP complex 240 is bound to the accumulating “chain” including SFLT-1, the remaining solution is discarded so that only SFLT-1 bound ALP remains. A pigmented test agent having an affinity for the color complex is added to generate a pigment indicative of the biomarker. For the second complex 240, an initially colorless ALP substrate 250 solution is added.

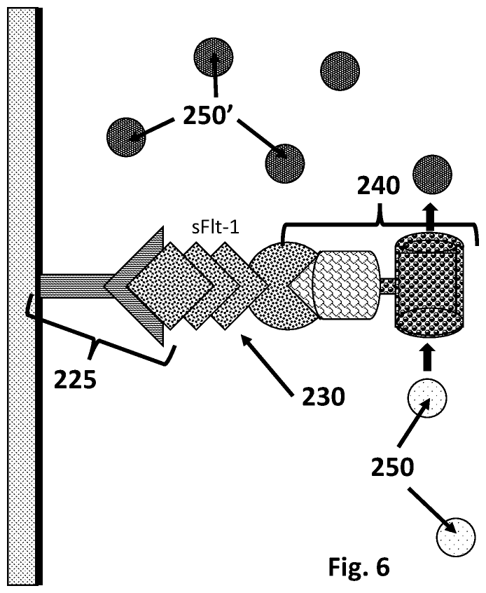

FIG. 6 shows binding of the pigmented test agent as in FIG. 5 to form a color factory defined by the ALP substrate; Over time, the ALP converts this colorless substrate into a vibrant blue pigment 250′. ALP substrates can be sourced for pigmented test agents of various colors. In the disclosed approach, a blue pigment is chosen for a visually contrasting combined shade with the yellow/orange creatinine reaction in the first containment 100. Various pigmented test agents and blended shades may be selected in alternate configurations.

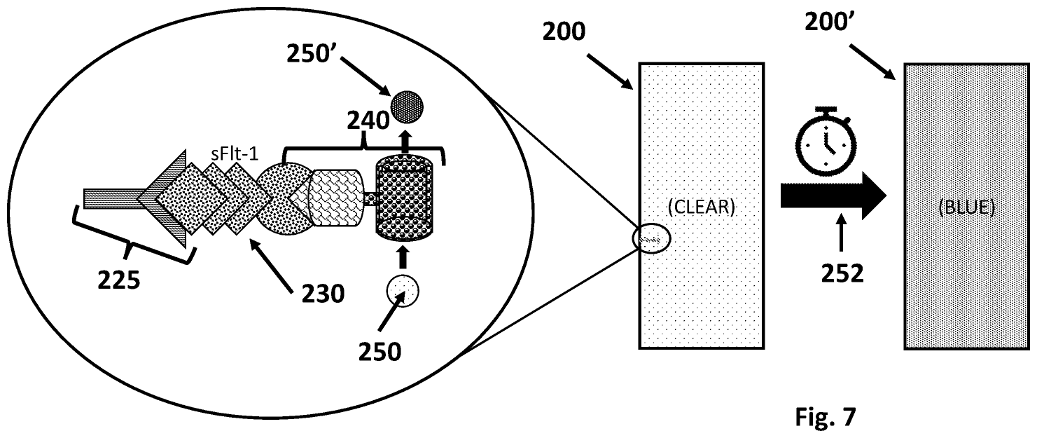

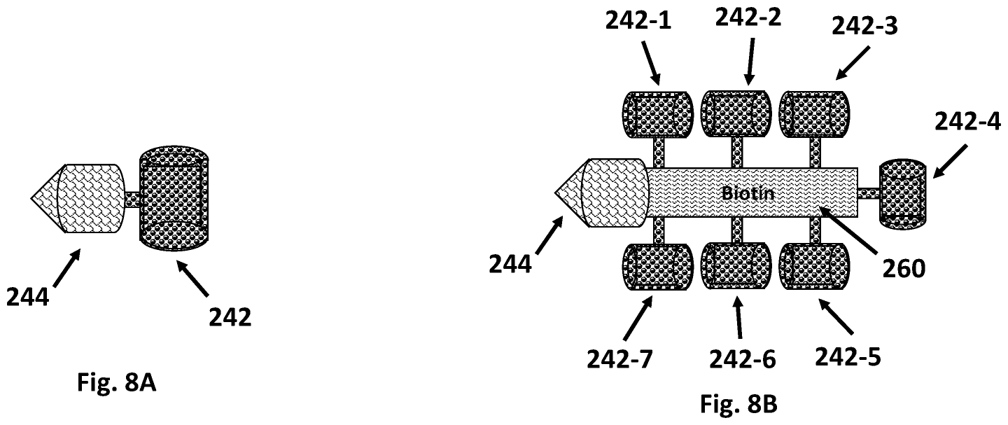

FIG. 7 shows pigment generation over time of the pigmented test agent of FIG. 6. The ALP substrate 250 results in a time sensitive reaction as the defined color factory continued to produce pigmented molecules 250′. After an elapsed interval for allowing the color complex to bind, the time-dependent blue pigment concentration is indicative of the SFLT-1 in the original urine sample. Referring to FIG. 8, the pigmented test agent reaction that produces pigment 250′ from the colorless ALP substrate 250 continues to a prescribed time limit 252 based on testing parameters. In the disclosed approach, after a given amount of time, the color of the reaction containment 200 will turn into a diagnostic shade proportional to the amount of SFLT-1 in from the solution of the first complex (FIG. 3), now bound to the containment sides. Alternate timing and/or color factory substates could be employed. The solution in the containment (now an ALP substrate pigment producing test agent) transitions the shade from an initial clear/colorless appearance in the containment 200 to a pigmented stage 200′, shown as blue in the disclosed approach.

FIGS. 8A and 8B show an alternate configuration using biotinylation for augmenting pigment generation in the pigmented test agent of FIGS. 6 and 7. The use of one alkaline phosphatase enzyme (ALP) per VEGF molecule may produce slow reaction times in FIG. 8A. This reaction rate can be increased by modifying the amount of ALP attached to each VEGF molecule via biotinylation as in FIG. 8B. Referring to FIG. 8A, an initial configuration binds a single ALP 242 to a VEGF 244 molecule for forming the second color complex 240-N, as in FIG. 4. The pigmented blue shade can be produced faster by introducing additional color complex 240 molecules, which ultimately each respond to the ALP substrate 250 for pigment production. FIG. 8B conjugates the subcomplex 240 via biotinylation 260 for forming a subcomplex including a plurality of ALP (alkaline phosphatase) 240′-1..240′-N molecules bound to a VEGF molecule.

Recall from above that a feature of the disclosed approach is for a fast, onsite testing product that combines the first and second containment 100, 200 in a combined vial structure suited for facilitated exchange of the test sequence and solution exchanges invoked for producing the respective shades 101, 201 in each of the adjacent, transparent containments 100, 200, and then viewing a combined shade against a color scale 310, once the test is complete. Referring to FIGS. 1-8B, the device 100 arranges a first containment 100 containing a first biomarker (for creatinine) adjacent to a second containment 200 for a second biomarker (for SFLT-1), where both containments are transparent and have a common optical axis for simultaneous viewing via a line of sight through both containments. To render the combined shade, the first and second containments are aligned such that the common optical axis is adjacent the color scale 310 indicative of a plurality of shades, where each shade is indictive of a ratio of the first biomarker and the second biomarker for mapping the combined shade 301 to a concentration of the first biomarker indicative of a diagnosis.

An example use case of the endometriosis test device is as follows:

1. Device is assembled as a packaged unit for distribution:
a. Complex 1 is fully assembled and attached to an inner surface of the SFLT-1 Analysis Tube (either on interior surface or attached to magnetic balls in the containment 200);
b. Complex 2 240 is present, uncoupled to Complex 1 (not yet joined with SFLT-1 linker), prepared in available tube or vial;
c. ALP Substrate included in additive vial;
d. Picric acid is either present in a Creatinine Analysis Tube (containment 100) or included as an additive for creatinine measurement;

2. Doctor/user purchases and opens device;

3. Urine sample from patient defines test specimen including both creatinine and SFLT-1 (if present) and added to both containments 100, 200;

4. Device 10 is capped and lightly shaken to mix the respective contents in the containments 100, 200. Urinary bound SFLT-1 binds to complex 1 225 antibodies;
a. creatinine reaction in containment 100 proceeds to stable yellow/orange color;
b. VEGF-ALP substrate (complex 2) either initially within or added to containment 2 for binding to SFLT-1;

5. Urine (along with uncoupled Complex 2) is poured out of SFLT-1 containment 200 so that remaining complex 2 is based on SFLT-1 presence in test specimen;

6. Containment 200 may be rinsed with saline (provided) to ensure only bound SFLT-1 remains;

7. ALP Substrate 250 additive poured into SFLT-1 containment 200; 8. Device 10 is left alone for a time period 252 (˜1 hour) to generate blue shade 201 proportional to SFLT-1 presence;

9. User returns to device and activates LED light on device for illuminating combined shade 301;

10. User looks through viewing window defining the common line of sight;
a. visually compares color to chart attached to window; OR
b. checks wavelength filter for light pass-through or full darkness of the combined shade 301;

Referring again to FIG. 1, the color of the SFLT-1 reaction tube (containment 200) can be viewed together with the color of the creatinine reaction (containment 100) tube to produce a combined diagnostic shade when viewed in series. This shade can then be compared to a color chart which indicates the presence and degree/quantity of SFLT-1 indicative of endometriosis diagnosis. The combined shade 301 results from the first biomarker (SFLT-1) rendering a shade having a wavelength corresponding to blue and the second biomarker (creatinine) rendering a shade having a wavelength corresponding to yellow.

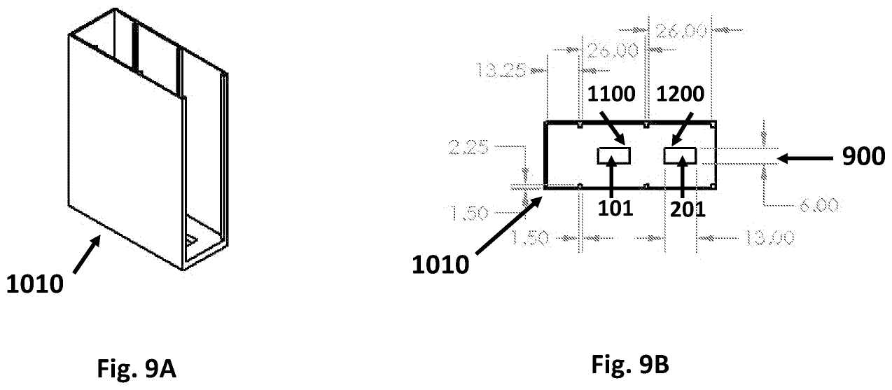

FIGS. 9A and 9B show an example of the device including the vials and color scale indicative of the endometriosis diagnosis result. Referring to FIGS. 1, 9A and 9B, the device 10 takes the form of a molded or printed case 1010, having bays or slots 1100, 1200 capable of retaining the containments 100, 200, respectively. Upon generation of the shades 101 and 201 in the respective containments as discussed above, a view along a line of sight 900 renders the viewable/perceptible blended shade 301. The color scale 310 for comparison may be embedded against the back wall of the device or may be a separate card or sheet immediately adjacent to the case 1010 for visual comparison..

While the system and methods defined herein have been particularly shown and described with references to embodiments thereof, it will be understood by those skilled in the art that various changes in form and details may be made therein without departing from the scope of the invention encompassed by the appended claims.

1. A method for detecting a presence of a biomarker, comprising:
coating a surface with a binding protein;
adhering an antibody of the biomarker to the binding protein;
combining a test specimen containing the biomarker with the antibody of the biomarker for binding the biomarker to the antibody;
conjugating a subcomplex including an ALP (alkaline phosphatase) bound VEGF (vascular endothelial growth factor) and combining the subcomplex with the bound test biomarker for forming a color complex;
adding a pigmented test agent having an affinity for the color complex to generate a pigment indicative of the biomarker; and
determining a presence of the biomarker based on a visual shade resulting from the generated pigment.

2. The method of claim 1 further comprising coating a containment with a binding protein for receiving a liquid of the test specimen.

3. The method of claim 1 further comprising first and second biomarkers, the second biomarker receptive to a pigmented test agent for generating a second pigment, further comprising:
generating the visual shade and a second shade from the second pigment in adjacent transparent containments; and
rendering a combined shade based on a line of sight through both of the adjacent transparent containments.

4. The method of claim 3 wherein the adjacent transparent containments are open for optical inspection for viewing the combined shade adjacent to a color scale indicative of a ratio of the first and second biomarkers.

5. A method for detecting a ratio of biomarkers, comprising:
generating a plurality of reactions in a respective plurality of containments, each reaction of the plurality of reactions based on a pigmented test agent indicative of a presence of a biomarker;
forming a shade in each of the containments, the shade resulting from a concentration of the respective biomarker; the containments transparent for visualization of the shade; and
disposing the containments in an optical adjacency for generating a combined shade based on each respective shade, the combined shade indicative of a ratio of each of the biomarkers.

6. The method of claim 5 wherein at least one of the shades results from:
coating a surface in one of the containments with a binding protein;
adhering an antibody of the biomarker to the binding protein;
combining a test specimen containing the biomarker with the antibody of the biomarker for binding the biomarker to the antibody;
binding a conjugate including a protein and a coloring enzyme to the bound test biomarker for forming a color complex, the color complex responsive to a pigmented test agent;
adding the pigmented test agent having an affinity for the color complex to generate a pigment indicative of the biomarker; and
determining a presence of the biomarker based on a visual shade resulting from the generated pigment.

7. The method of claim 6 wherein binding the conjugate further comprises: conjugating a subcomplex including an ALP (alkaline phosphatase) bound VEGF (vascular endothelial growth factor); and
combining the subcomplex with the bound test biomarker for forming the color complex.

8. The method of claim 6 further comprising:
following an interval for allowing the biomarkers in the test specimen to bind with the antibody, removing the remaining test specimen from the containment.

9. The method of claim 6 further comprising:
binding the conjugate by adding a solution including the color complex to the containment; and
after an elapsed interval for allowing the color complex to bind to the previously bound biomarker, removing the added color complex solution to remove unbound conjugate and avoid subsequent binding with the pigmented test agent.

10. The method of claim 6 wherein at least one of the shades results from:
combining an acidic indicator with the test specimen in an alkaline environment, the acidic indicator responsive to a presence of a second biomarker in the test specimen.

11. The method of claim 10 wherein the acidic indicator is picric acid and the second biomarker is creatinine.

12. The method of claim 7 further comprising
conjugating the subcomplex via biotinylation for forming a subcomplex including a plurality of ALP (alkaline phosphatase) molecules bound to a VEGF molecule.

13. The method of claim 5 further comprising:
arranging a first containment of the plurality of containments for a first biomarker adjacent to a second containment for a second biomarker, both containments being transparent and having a common optical axis; and
orienting the first and second containments for aligning the common optical axis adjacent a color map indicative of a plurality of shades, each shade of the plurality of shades indictive of a ratio of the first biomarker and the second biomarker for mapping the combined shade to a concentration of the first biomarker indicative of a diagnosis.

14. The method of claim 13 wherein the first biomarker is SFLT-1 and the second biomarker is creatinine.

15. The method of claim 13 wherein the first biomarker renders a shade having a wavelength corresponding to blue and the second biomarker renders a shade having a wavelength corresponding to yellow.

16. A genetic screening test device, comprising:
a first vessel including a pigmented test agent for a first biomarker;
a second vessel including a pigmented test agent for a second biomarker, the first and second vessels visually aligned for a common line of sight; and
a predetermined range of rendered colors, the rendered colors based on a combined shade resulting from a respective percentage of the first and second biomarkers.

17. The device of claim 16 wherein each of the pigmented test agents is indicative of a concentration of a biomarker.

18. The device of claim 16 wherein the pigmented test agents are responsive to a respective biomarker for generating a pigmented shade having a color depth relative to the biomarker concentration.

19. The device of claim 16 wherein the combined shade results from a blend of colors in a line of sight common to the first vessel and the second vessel.

20. The device of claim 16 wherein the first vessel and the second vessel are transparent and adjacent for rendering a blended color based on a concurrent view of both the first and second vessel in a common line of sight, further comprising an adjacent background and rendering of the predetermined range of rendered colors, the background adapted for simultaneous presentation of the blended color against the predetermined range of rendered colors.
